(19) United States
(12) Patent Application Publication
(10) Pub. No.: US 2023/0284989 A1
(43) Pub. Date: Sep. 14, 2023

(54) MOBILE RADIOGRAPHIC IMAGING APPARATUS, DYNAMIC IMAGE OUTPUT METHOD AND STORAGE MEDIUM

(71) Applicant: Konica Minolta, Inc., Tokyo (JP)

(72) Inventors: Yuuichi NISHIJIMA, Tokyo (JP); Ichirou HAMAMOTO, Tokyo (JP); Naoki HAYASHI, Tokyo (JP)

(21) Appl. No.: 18/176,919

(22) Filed: Mar. 1, 2023

(30) Foreign Application Priority Data
Mar. 8, 2022 (JP) 2022-034900

(51) Int. Cl. A61B 6/00

(52) U.S. Cl. CPC A61B 6/463; A61B 6/5205; A61B 6/563

(57) ABSTRACT

A mobile radiographic imaging apparatus is capable of performing dynamic imaging of radiographic imaging and includes a hardware processor. The hardware processor wirelessly outputs a first dynamic image including a plurality of frames obtained by the dynamic imaging to an external apparatus, and after obtaining at least two frames of the plurality of frames, wirelessly outputs a second dynamic image for display based on the first dynamic image to an external display apparatus.

### REFERENCE TO RELATED APPLICATIONS

The entire disclosure of Japanese Patent Application No. 2022-034900, filed on Mar. 8, 2022, including description, claims, drawings and abstract is incorporated herein by reference in its entirety.

### TECHNICAL FIELD

The present disclosure relates to a mobile radiographic imaging apparatus, a dynamic image output method and a storage medium.

### DESCRIPTION OF THE RELATED ART

It is known that a nursing cart (mobile radiographic imaging apparatus) that performs dynamic imaging with radiation, thereby obtaining a dynamic image composed of a plurality of frames, transmits the dynamic image by wireless communication. (See, for example, JP 2018-7851 A.)

Dynamic imaging using such a nursing cart during a surgical operation is, for example, for checking the route of a catheter or a tube during the surgical operation or for checking whether no object (gauze) is left in the body of the patient at the end of the surgical operation. Since the monitor screen of such a nursing cart is relatively small, it is desired that during a surgical operation, dynamic images be checked using an external display apparatus having a large monitor screen.

If an image(S) to be checked is a fluoroscopic image(s) obtained by a fluoroscopic apparatus, it can be displayed on an external display apparatus in real time, but since a dynamic image is a package of a plurality of frames, it cannot be output to any external apparatus, including an external display apparatus, until image processing for output is performed on all frames thereof and all frames for output are generated.

In order to minimize an exposure dose, the frame rate of dynamic imaging tends to be low (e.g., 7-15 fps) within a range in which dynamic analysis of dynamic images about ventilation, blood flow or the like can be performed properly. Since this frame rate is lower than the frame rate of general video playback (e.g., 30-60 fps), a dynamic image output to and played on an external display apparatus cannot not be played smoothly, and a doctor or/or others who watch this feel something strange.

Meanwhile, it is desired to output dynamic images obtained by dynamic imaging to a picture archiving and communication system (PACS) for storage as evidence and/or to an external dynamic analysis apparatus for dynamic analysis, such as ventilation analysis or blood flow analysis. In consideration of output to the abovementioned external apparatuses, if a dynamic image is output to such an external apparatus and then output from the external apparatus to an external display apparatus, it takes a considerable amount of time before the dynamic image can be checked on the external display apparatus.

### SUMMARY OF THE INVENTION

The present disclosure has been made in view of the above problems, and objects thereof include allowing a doctor and/or other(s) (user(s)) to promptly check a dynamic image on an external display apparatus with the feeling of strangeness reduced.

To achieve at least one of the abovementioned objects, according to a first aspect of the present disclosure, there is provided a mobile radiographic imaging apparatus capable of performing dynamic imaging of radiographic imaging, including a hardware processor that

wirelessly outputs a first dynamic image including a plurality of frames obtained by the dynamic imaging to an external apparatus, and

after obtaining at least two frames of the plurality of frames, wirelessly outputs a second dynamic image for display based on the first dynamic image to an external display apparatus.

To achieve at least one of the abovementioned objects, according to a second aspect of the present disclosure, there is provided a dynamic image output method that is performed by a mobile radiographic imaging apparatus capable of performing dynamic imaging of radiographic imaging, including:

wirelessly outputting a first dynamic image including a plurality of frames obtained by the dynamic imaging to an external apparatus; and

after obtaining at least two frames of the plurality of frames, wirelessly outputting a second dynamic image for display based on the first dynamic image to an external display apparatus.

To achieve at least one of the abovementioned objects, according to a third aspect of the present disclosure, there is provided a non-transitory computer-readable storage medium storing a program that causes, of a mobile radiographic imaging apparatus capable of dynamic imaging of radiographic imaging, a computer to:

wirelessly output a first dynamic image including a plurality of frames obtained by the dynamic imaging to an external apparatus; and

after obtaining at least two frames of the plurality of frames, wirelessly output a second dynamic image for display based on the first dynamic image to an external display apparatus.

### BRIEF DESCRIPTION OF THE DRAWINGS

The advantages and features provided by one or more embodiments of the present disclosure will become more fully understood from the detailed description given hereinafter and the appended drawings, which are given by way of illustration only, and thus are not intended as a definition of the limits of the present disclosure, wherein:

FIG. 1 shows an example of the overall configuration of a radiographic imaging system;

FIG. 2 is a block diagram showing a functional configuration of a mobile radiographic imaging apparatus shown in FIG. 1;

FIG. 3 is a flowchart of an image output control process that is performed by a controller shown in FIGS. 2; and

FIG. 4 shows an example of a cine display screen that is displayed on a display in Step S4 shown in FIG. 3.

### DETAILED DESCRIPTION

Hereinafter, one or more embodiments of the present disclosure will be described with reference to the drawings. However, the technical scope of the present disclosure is not limited to the following embodiments or illustrated examples.

<Configuration of Radiographic Imaging System 100>

First, configuration of an embodiment(s) of the present disclosure will be described.

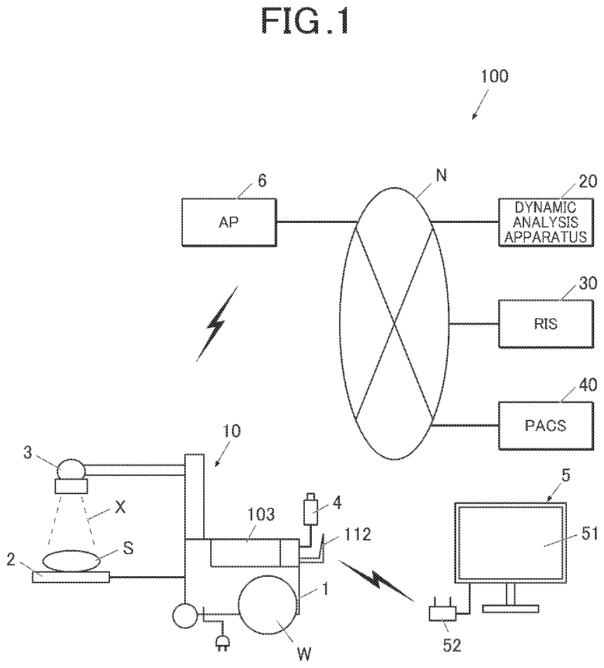

FIG. 1 shows an example of the overall configuration of a radiographic imaging system 100 of an embodiment(s).

The radiographic imaging system 100 is a system constructed in a medical facility, and as shown in FIG. 1, configured such that a mobile radiographic imaging apparatus 10, a dynamic analysis apparatus 20, a radiology information system (RIS) 30 and a picture archiving and communication system (PACS) 40 are connected to one another via a communication network N, such as a local area network (LAN) or a wide area network (WAN), in the medical facility so that they can transmit and receive data to and from one another. In the medical facility where the radiographic imaging system 100 is installed, a plurality of wireless access points (APs) 6 is provided. The mobile radiographic imaging apparatus 10 is connectable to the communication network N via the wireless access point(s) 6. The apparatuses connected to the communication network N conform to the digital image and communications in medicine (DICOM) standard Hence, communications between the apparatuses via the communication network N are performed in conformity to the DICOM standard

The mobile radiographic imaging apparatus 10 can transmit and receive data to and from an external display apparatus 5 via a general-purpose wireless LAN that is different from the hospital communication network N.

The mobile radiographic apparatus 10 is, for example, an apparatus that performs radiographic imaging of patients during a round of visits, the patients having difficulty in moving. The mobile radiographic imaging apparatus 10 includes a body 1 having wheels W, and is configured as a mobile nursing cart. The mobile radiographic imaging apparatus 10 may be portable without a wheel.

The mobile radiographic imaging apparatus 10 is brought into an operating room, an intensive care unit (ICU), a room in a ward or the like, and emits radiation from a radiation source 3 in a state in which an FPD 2 is inserted, for example, between a subject S who lies down on a bed and the bed or into a not-shown insertion port provided on a side of the bad opposite to a side where the subject S lies down, thereby performing still imaging or dynamic imaging of the subject S. In this embodiment, still imaging refers to obtaining one image of the subject S in response to a single imaging operation, and dynamic imaging (kinetic imaging) refers to obtaining a plurality of images of the subject S in response to a single imaging operation by repeatedly emitting pulsed radiation, such as X-rays, to the subject S at intervals of a predetermined time (pulse emission) or continuously emitting radiation without a break to the subject S at a low dose rate (continuous emission). A series of images obtained by (a series of steps of) dynamic imaging is called a dynamic image. Images constituting a dynamic image are called frame images or frames.

Dynamic imaging includes video (moving image) shooting, but does not include taking still images while displaying a video. Dynamic images include videos, but do not include images obtained by performing still imaging while displaying a video.

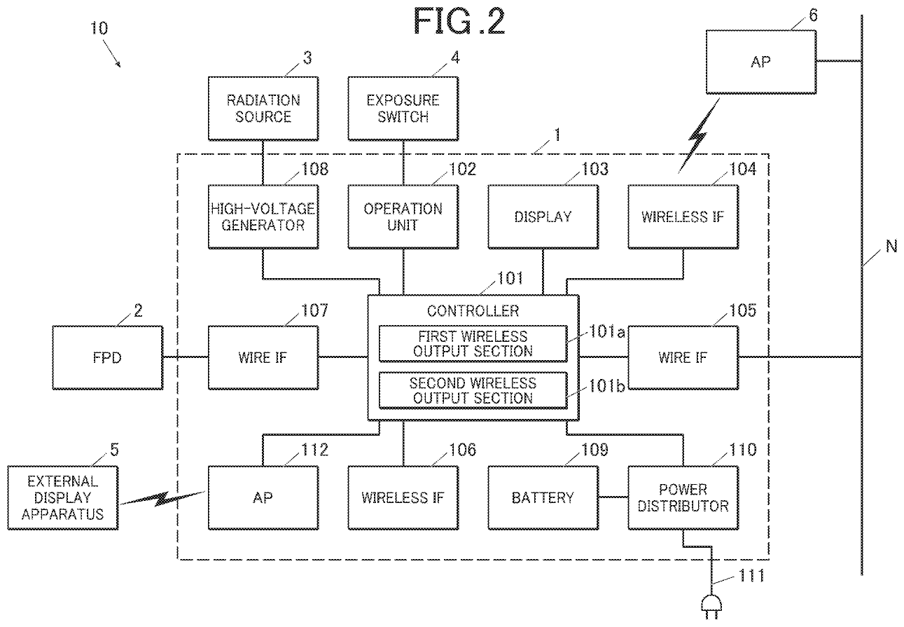

FIG. 2 is a block diagram showing a functional configuration of the mobile radiographic imaging apparatus 10.

As shown in FIG. 2, the mobile radiographic imaging apparatus 10 includes the body 1, the FPD 2, the radiation source 3 and an exposure switch 4.

The body 1 includes a controller 101 (hardware processor), an operation unit 102, a display 103, a wireless IF 104, a wire IF 105, a wireless IF 106, a wire IF 107, a high-voltage generator 108, a battery 109, a power distributer 110, and a wireless access point (AP) 112.

The controller 101 includes a central processing unit (CPU), a random access memory (RAM), and a read only memory (ROM). The CPU of the controller 101 reads various programs stored in the ROM, loads the read programs to the RAM, and performs various processes in accordance with the loaded programs, thereby performing centralized control of operation of the components of the mobile radiographic imaging apparatus 10.

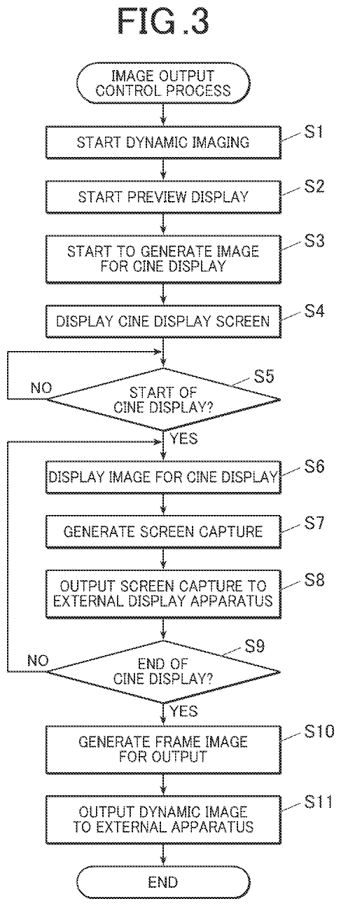

In this embodiment, the controller 101 functions as a first wireless output section 101a and a second wireless output section 101b by performing an image output control process (shown in FIG. 3), which is described later, in cooperation with a program stored in the ROM. The first wireless output section 101a wirelessly outputs a dynamic image (first dynamic image) including a plurality of frames obtained by dynamic imaging to an external apparatus (dynamic analysis apparatus 20 or PACS 40). After obtaining at least two frames of the frames of the dynamic image, the second wireless output section 101b wirelessly outputs a dynamic image for display (second dynamic image) based on the dynamic image to the external display apparatus 5.

The operation unit 102 includes operation buttons and a touchscreen, and detects content of an operation made by a user, such as a type of an operation button pressed or a point touched with a finger or a touch pen, and outputs same to the controller 101 as operation information.

The exposure switch 4 for the user to make an instruction to emit radiation X is connected to the operation unit 102.

The exposure switch 4 may be configured to be remotely operable by being wirelessly or wire connected to the mobile radiographic imaging apparatus 10. In this manner, the user can control radiation exposure (radiation emission) away from the body 1 of the mobile radiographic imaging apparatus 10.

The display 103 is constituted by a monitor, such as a liquid crystal display (LCD) or a cathode ray tube (CRT), and displays examination order information and images in accordance with instructions of display signals input from the controller 101.

Examination order information is transmitted, for example, from the RIS 30, and includes examination identification information (examination ID, etc.), an examination date, patient information on a patient who is the subject S (patient ID, name, sex, age, room in hospital (ward, operating room, etc.), etc.), and information on each imaging included in the examination (imaging ID, imaging type, i.e., still imaging or dynamic imaging, imaging conditions (imaging site, imaging direction, frame rate, number of images, imaging time, emission power of radiation source 3, etc.), type of analysis to be performed by dynamic analysis apparatus 20 (type or name of analysis, etc.), requesting unit, imaging location (room in ward, operating room, ICU, etc.), etc.).

The wireless IF 104 is an interface that is wirelessly connected to the wireless access point(s) 6 to transmit and receive (output and input) data to and from an external apparatus(es) (dynamic analysis apparatus 20, RIS 30, PACS 40, etc.) connected to the communication network N via the wireless access point(s) 6.

The wire IF 105 is an interface into which a communication cable is inserted to be connected to the communication network N by wire communication to transmit and receive data to and from an external apparatus connected to the communication network N.

The connection to the communication network N can switch between wire connection and wireless connection in response to control signals from the controller 101.

The wireless IF 106 is a wireless interface to transmit and receive data to and from the FPD 2 and/or the external display apparatus 5 connected to a general-purpose communication network, such as a wireless LAN, by wireless communication via the wireless access point 112. In this embodiment, the body 1 of the mobile radiographic imaging apparatus 10 includes the wireless IF 106. However, if the wireless access point 112 and the controller 101 are wire connected, the wireless IF 106 is unnecessary.

The wire IF 107 is an interface into which a communication cable is inserted to transmit and receive data to and from the FPD 2 by wire communication.

The connection to the FPD 2 can switch between wire connection and wireless connection in response to control signals from the controller 101.

The high-voltage generator 108 applies, to the radiation source 3, a voltage suitable for preset radiation emission conditions (imaging conditions on radiation emission, such as imaging type, i.e., dynamic imaging or still imaging, tube voltage, tube current, emission time, and product of current and time) in response to receiving a control signal from the controller 101.

The battery 109 can supply electric power stored in itself to the power distributor 110, and also can store electric power supplied from the power distributor 110.

The power distributor 110 has a power cable 111 provided with a plug at its tip, and can receive electric power from the outside with the plug inserted in a nearby socket. The power distributor 110 distributes electric power supplied from the battery 109 or the outside to the components of the mobile radiographic imaging apparatus 10.

The wireless access point (AP) 112 is a wireless access point provided in the mobile radiographic imaging apparatus 10. Via the wireless access point 112, the wireless IF 106 and the external display apparatus 5 and/or the FPD 2 transmit and receive data to and from one another by wireless communication.

The FPD 2 includes a substrate, a reading circuit, a controller, a communication unit and a connector. On the substrate, pixels provided with radiation detection elements and switch elements are arranged two-dimensionally (in a matrix). The radiation detection elements generate electric charges corresponding to a dose of received radiation X. The switch elements accumulate and release the electric charges. The reading circuit reads the amounts of the electric charges released from the respective pixels as signal values. The controller generates image data from the signal values read by the reading circuit. The communication unit transmits the image data and various signals to the body 1 by wire or wireless communication. The connector is the one into which a cable is inserted to be connected to the body 1.

The FPD 2 may have a built-in scintillator or the like and convert received radiation X into light having another wavelength, such as visible light, with the scintillator, and generate electric charges corresponding to the light obtained by the conversion, which is called indirect type, or may generate electric charges directly from received radiation X without a scintillator or the like, which is called direct type.

The radiation source 3 has, for example, a rotating anode and a filament, which are not shown. When the high-voltage generator 108 applies a voltage to the radiation source 3, the filament irradiates the rotating anode with an electron beam corresponding to the voltage, and the rotating anode generates a dose of radiation X corresponding to the intensity of the electron beam.

The external display apparatus 5 includes a monitor 51 having a large screen (larger than the screen of the display 103), such as an LCD, and a wireless IF 52. Data of a dynamic image(s) for display transmitted from the mobile radiographic imaging apparatus 10 via the wireless access point 112 is received by the wireless IF 52 and displayed on the monitor 51. As the external display apparatus 5, a monitor of a general-purpose product, namely a general-purpose monitor, is usable. The external display apparatus 5 is disposed, for example, near the mobile radiographic imaging apparatus 10 in a room, such as an operating room, where the mobile radiographic imaging apparatus 10 is performing dynamic imaging.

The dynamic analysis apparatus 20 analyzes a dynamic image(s) output from the mobile radiographic imaging apparatus 10, and transmits the dynamic image and the analysis result to the PACS 40. The dynamic analysis apparatus 20 can perform multiple types of analysis and performs a type(s) of analysis specified from among the multiple types of analysis.

The RIS 30 is an order issuing apparatus that issues and stores examination order information, and also transmits the issued examination order information to the mobile radiographic imaging apparatus 10 via the communication network N.

The PACS 40 is an image management apparatus that correlates, stores and manages each medical image (still image, dynamic image) generated by a modality, such as the mobile radiographic imaging apparatus 10, and an analysis result thereof generated by the dynamic analysis apparatus 20 with patient information and examination information.

<Operation>

Next, operation of the mobile radiographic imaging apparatus 10 will be described.

In the mobile radiographic imaging apparatus 10, when receiving examination order information from the RIS 30 through the wireless IF 104 or the wire IF 105, the controller 101 stores the received examination order information in the RAM, and causes the display 103 to display the received examination order information in an examination list screen (not shown).

When examination order information on a target examination is selected by a user from the examination list screen using the operation unit 102, the controller 101 causes the display 103 to display a button(s) for selecting imaging included in the examination order information to allow the user to select imaging to be performed. When the imaging to be performed is selected, the controller 101 sets, to the high-voltage generator 108, the emission conditions (tube current, tube voltage, emission time, product of current and time, etc.) based on content of an order (request) for the selected imaging, and also sets reading conditions to the FPD 2.

When the exposure switch 4 is pressed, the controller 101 controls the high-voltage generator 108 and the FPD 2 to start radiographic imaging (still imaging or dynamic imaging).

The mobile radiographic imaging apparatus 10 may perform dynamic imaging during a surgical operation for a doctor and/or others (i.e., user(s)) to check the route of a catheter or a tube during the surgical operation or to check whether no object (gauze) is left in the body of the patient at the end of the surgical operation. Considering that a plurality of persons may watch dynamic images during a surgical operation, and the monitor of the display 103 included in the mobile radiographic imaging apparatus 10 is relatively small, it is preferable that dynamic images be displayed on the large screen of the external display apparatus 5.

However, dynamic images are conventionally transmitted from mobile radiographic imaging apparatuses to external apparatuses by communications conforming to the DOCOM standard In the DICOM standard, a dynamic image is treated as one file, namely, a complete set of frame images of a dynamic image is treated as one package/file. Hence, output of a dynamic image cannot be started until all frame images, which are obtained by a series of steps of dynamic imaging (imaging that starts with an instruction to start dynamic imaging being made, for example, by pressing the exposure switch 4, and finishes with the instruction being lifted (terminated), for example, by releasing the exposure switch 4), are ready (obtained). Hence, it takes time before the output.

Dynamic imaging may be set at a low frame rate (e.g., 7-15 fps) in order to reduce an exposure dose of a patient. This frame rate is lower than the frame rate of general video playback. Hence, output of a dynamic image to (display thereof on) the monitor of the external display apparatus 5 at the set frame rate results in no-smooth video playback, and a doctor and/or others who watch the dynamic image feel something strangeness about the dynamic image.

The mobile radiographic imaging apparatus 10 of this embodiment therefore includes, in addition to the first wireless output section 101a, which in conformity to the DICOM standard, outputs a dynamic image including a plurality of frame images obtained by a series of steps of dynamic imaging to an external apparatus (dynamic analysis apparatus 20 or PACS 40), the second wireless output section 101b, which after obtaining at least two frame images by the dynamic imaging, outputs a dynamic image for display based on the dynamic image to the external display apparatus 5 by wireless communication. This allows a doctor and/or others to promptly check a dynamic image on the external display apparatus 5 with the feeling of strangeness reduced.

FIG. 3 is a flowchart of the image output control process that is performed in the mobile radiographic imaging apparatus 10. The image output control process is performed by the CPU of the controller 101 in cooperation with a program stored in the ROM of the controller 101 when dynamic imaging is selected as target imaging and the exposure switch 4 is pressed to makes an instruction to start dynamic imaging Hereinafter, the image output control process will be described with reference to FIG. 3.

This embodiment is described as a case where the FPD 2 is already connected to the mobile radiographic imaging apparatus 10 via the wire IF 107, and wireless communications between the wireless IF 106 and the external display apparatus 5 are already established via the wireless access point 112. However, wireless communications between the wireless IF 106 and the external display apparatus 5 may be established, after start of dynamic imaging, at a predetermined timing that is before start of output of a dynamic image for display. The wireless access point(s) 6 and the wireless IF 104 may double as the wireless access point 112 and the wireless IF 106, respectively, or if appropriate, vice versa, and the controller 101 functions as the first wireless output section 101a or the second wireless output section 101b by its own determination, thereby switching connection destinations.

The controller 101 first transmits an instruction to start dynamic imaging to the high-voltage generator 108 and the FPD 2 so that they start a series of steps of dynamic imaging (Step S1).

In dynamic imaging, in order to reduce an exposure dose of a patient, imaging is performed at a lower frame rate (e.g., 7-15 fps) than that of general video playback (e.g., 30-60 fps) by the radiation source 3 emitting a lower dose of radiation to the subject S than that in still imaging. The FPD 2 sequentially transmits frame images obtained by dynamic imaging to the body 1.

When the exposure switch 4 is released, the controller 101 transmits an instruction to finish dynamic imaging to the high-voltage generator 108 and the FPD 2 so that they finish the series of steps of dynamic imaging. The controller 101 proceeds to Step S2 without waiting for the end of the series of steps of dynamic imaging.

When starting to receive, through the wire IF 107, frame images of the dynamic image transmitted from the FPD 2, the controller 101 correlates and temporarily stores each received frame image with a frame number in the RAM, and also causes the display 103 to start preview display of sequentially displaying the received frame images (Step S2).

Preview display is displaying frame images obtained by dynamic imaging for tentative checking, for example, at a low frame rate that is the same as the frame rate of the dynamic imaging.

During preview display, the controller 101 starts to generate images for cine display based on the respective received frame images (Step S3).

Cine display of a dynamic image refers to continuously and sequentially displaying (a series of) frame images of a dynamic image (image-processed dynamic image included), namely playing a video. As described above, since dynamic images are images obtained with a low dose of radiation, they have a large amount of noise content, and therefore are difficult to watch for persons if the display 103 or the external display apparatus 5 cine-displays dynamic images as they are. Hence, in Step S3, the controller 101 performs image processing on the received frame images to remove noise content, thereby generating images for cine display of the dynamic image. Examples of the image processing for removing noise content include averaging and moving average processing using a plurality of frame images (frame images adjacent in terms of time), but not limited thereto. Other image processing, such as gradation processing and/or frequency enhancement, may also be performed.

The controller 101 causes the display 103 to display a cine display screen 130 (Step S4).

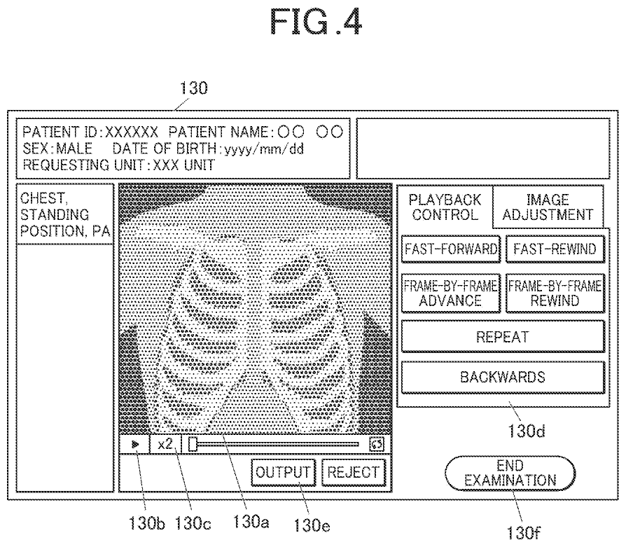

For example, during preview display on the display 103, the controller 101 causes the display 103 to display an operation button or the like for making an instruction to move to the cine display screen 130 (shown in FIG. 4). For example, the controller 101 causes the display 103 to display the operation button at the timing when cine display of frame images that are obtained by dynamic imaging becomes performable by the controller 101 generating, as to all the frame images, images for cine display based on the respective frame images. As another example, if the CPU of the controller 101 is a very-high-performance CPU and can generate images for cine display faster than the speed of cine display, the controller 101 causes the display 103 to display the operation button for making an instruction to move to the cine display screen 130 as soon as the controller 101 starts to receive frame images obtained by dynamic imaging and starts to generate images for cine display. As another example, if the CPU of the controller 101 is a not-so-high-performance CPU and generates images for cine display slower than the speed of ine display, the controller 101 causes the display 103 to display the operation button for making an instruction to move to the cine display screen 130 when the remaining number of images for cine display to generate becomes equal to or less than a predetermined number (or after the controller 101 generates images for cine display of all the frame images). As described above, cine display of a dynamic image at a frame rate equal or similar to that of dynamic imaging by which the dynamic image has been obtained results in no-smooth playback, and a doctor and/or others who watch the dynamic image feel something strange about the dynamic image. Considering that cine display may be performed at double the frame rate of dynamic imaging, the controller 101 causes the display 103 to display the operation button for making an instruction to move to the cine display screen 130 at a proper timing. When the displayed operation button is pressed, the controller 101 causes the display 103 to display the cine display screen 130. Further, when preview display finishes, the controller 101 automatically causes the display 103 to display the cine display screen 130.

FIG. 4 shows an example of the cine display screen 130. As shown in FIG. 4, for example, the cine display screen 130 includes (displays) an image display area 130a for displaying images for cine display, a play button 130b for making an instruction to start cine display (video playback), a fast-forward button 130c for making an instruction to perform cine display at double speed (2×), and a playback control area 130d for a variety of control for cine display. When the play button 130b is pressed, the button changes to a pause button. Not only the fast-forward button 130c for double speed but also fast-forward buttons for triple speed (3×) and so forth may be provided (displayed). A fast-forward instructing operation may be able to be made by specifying a frame rate other than a multiple of the frame rate of dynamic imaging, such as 2× or 3×. Further, in accordance with the frame rate for fast-forward, normal display time corresponding to the actual imaging time and shortened display time due to the frame rate for fast forward may be displayed in comparison with one another or individually.

The cine display screen 130 further includes (displays) an output button 130e for making an instruction to output a received dynamic image to an external apparatus (dynamic analysis apparatus 20 or PACS 40) and an examination end button 130f for making an instruction to end an examination.

When the play button 130b or the fast-forward button 130c is pressed to make an instruction to start cine display of the dynamic image obtained by the series of steps of dynamic imaging (Step S5; YES), the controller 101 starts a process of cine-displaying the dynamic image on the display 103, and also starts to generate and output screen captures as a dynamic image for display to the external display apparatus 5.

That is, first, the controller 101 causes the display 103 to display the images for cine display in the cine display screen 130 (Step S6).

In Step S6, a marker(s) (arrow(s) included) and/or a stamp(s) may be superimposed on the displayed image(s). Examples of information represented by the markers and stamps that are displayed on the displayed image(s) include information indicating the imaging direction (P→A, A→P, etc.), information indicating left or right, information indicating each imaging condition (tube current, tube voltage, product of current and time, frame rate, etc.), and information indicating a point of interest. However, if these pieces of information are displayed during cine display, flickering occurs, which makes the images for cine display difficult to watch for a doctor and/or others. Hence, the controller 101 causes the display 103 not to display such markers and stamps during cine display but to display these during suspension of cine display.

Next, in sync with the cine display (in sync with the images for cine display being displayed/played), the controller 101 generates screen captures of the cine display screen 130 (Step S7).

Then, the controller 101 as the second wireless output section 101b outputs (transmits) the generated screen captures to the external display apparatus 5 through the wireless IF 106 (Step S8). Until cine display finishes (Step S9; NO), the controller 101 repeats Steps S6-S8 while causing the display 103 to change the images for cine display to display in order of imaging (in order of frame numbers) at a frame rate corresponding to the pressed operation button (play button 130b or fast-forward button 130c).

In Step S7, the controller 101 may generate screen captures by capturing the entire screen 130 or by capturing a part of the screen 130, the part where the images are displayed, such as the image display area 130a. The controller 101 may generate capture images internally from the images for cine display used for the display in Step S6, instead of newly generating screen captures. Further, the controller 101 may predict, on the basis of the size and the number of the images for cine display, the total data amount in the case where the entire screen is captured, and if the total data amount exceeds a preset threshold value, generate screen captures by capturing only a partial area of the screen. Even if screen captures are generated by capturing only a partial area of the screen, information necessary for preventing wrong identification of a patient on the external display apparatus 5, such as patient information and examination information, may be added to the screen captures.

If the play button 130b is pressed in Step S5, the controller 101 causes the display 103 to perform cine display in the cine display screen 130 at the same frame rate as that of the dynamic imaging, and in sync with the cine display, generates screen captures at the same frame rate as that of the dynamic imaging in Steps S6-S7, and as the second wireless output section 101b, outputs (transmits) the screen captures to the external display apparatus 5. The external display apparatus 5 displays, on the monitor 51, the dynamic image for display received from the mobile radiographic imaging apparatus 10 at a frame rate approximately the same as that of the dynamic imaging. If the fast-forward button 130c in the cine display screen 130 is pressed in Step S5, the controller 101 causes the display 103 to perform cine display in the cine display screen 130 at double the frame date of the dynamic imaging, and in sync with the cine display, generates screen captures at double the frame rate of the dynamic imaging in Steps S6-S7, and as the second wireless output section 101b, outputs (transmits) the screen captures to the external display apparatus 5. The external display apparatus 5 displays the dynamic image for display at a frame rate approximately double the frame rate of the dynamic imaging. That is, the external display apparatus 5 can display the dynamic image for display at approximately the same frame rate as that of the cine display on the display 103. The user presses the fast-forward button 130c for cine display at double speed, and accordingly can promptly check a dynamic image on the large screen of the external display apparatus 5 at a frame rate equal or similar to that of general video playback without feeling something strange about the dynamic image. Further, for a point (point of time) that needs to check closely, the user presses the play button 130b for cine display at normal speed (normal frame rate equal or similar to that of the dynamic imaging), and accordingly can check the point attentively.

The process of generating, as a dynamic image for display, screen captures at the frame rate of cine display is a process with a high processing load. Hence, the controller 101 may perform control not to generate screen captures during dynamic imaging, for example, by causing the display 103 not to display the cine display screen 130 or making the play button 130b and the fast-forward button 130c unable to be pressed (disabling these buttons) so that the display 103 cannot start cine display. This can reduce the processing load during dynamic imaging and enables appropriate dynamic imaging. If the CPU of the controller 101 is a high-performance CPU, and generation of screen captures during a series of steps of dynamic imaging does not affect the dynamic imaging, the controller 101 may generate screen captures during a series of steps of dynamic imaging by allowing the user to press the play button 130b or the fast-forward button 130c during the series of steps of dynamic imaging. This can move up output and display of a dynamic image for display to and on the external display apparatus 5.

When cine display of the dynamic image finishes (Step S9; YES), the controller 101 performs image processing for output to an external apparatus on the frame images of the dynamic image obtained by the series of steps of dynamic imaging, thereby generating frame images for output (frames for output) of the dynamic image (Step S10).

The image processing for output to an external apparatus is, for example, a process of performing a predetermined type(s) of image processing, such as gradation processing, frequency enhancement and/or noise removal, on all the frame images of the dynamic image, thereby generating a series of frame images for output, and generating a dynamic image file in the DICOM format including the generated series of frame images for output.

After the image processing for output finishes, when the output button 130e is pressed to make an instruction to output the dynamic image to an external apparatus, or when the examination end button 130f is pressed to make an instruction to end the examination, the controller 101 as the first wireless output section 101a transmits the processed dynamic image (dynamic image file in the DICOM format) to the dynamic analysis apparatus 20 or the PACS 40 through the wireless IF 104 (Step S11), and ends the image output control process.

<Modification of Operation>

In the above-described image output control process, the image processing for output to an external apparatus is performed after output of a dynamic image for display to the external display apparatus 5. However, the image processing for output can be started at any time as far as it is after start of reception of a series of frame images of a dynamic image from the FPD 2. However, since a complete set of frame images of a dynamic image obtained by a series of steps of dynamic imaging is treated as one package/file in the DICOM standard, the image processing for output cannot be completed until all frame images of a dynamic image are ready. Therefore, output of a dynamic image to an external apparatus in response to a press on the output button 130e can be performed after all frame images obtained by a series of steps of dynamic image are ready and all frame images for output are generated.

On the other hand, output of a dynamic image for display to the external display apparatus 5 with a frame rate increased can be performed at any time as far as it is after at least two of frame images of a dynamic image are obtained and a dynamic image for display of the at least two frame images is generated. This allows a doctor and/or others to promptly check a dynamic image on the external display apparatus 5 with the feeling of strangeness reduced.

If the image processing for output is started immediately after start of reception of frame images of a dynamic image from the FPD 2, output of a dynamic image to an external apparatus can be performed at any time as far as it is after the image processing is performed on all the frame images and a dynamic image file thereof in the DICOM format is generated. Therefore, output of a dynamic image to an external apparatus in response to a press on the output button 130e can be performed even during a period from cine display to output of a dynamic image for display as far as it is after the dynamic image file is generated. Further, cine display may be started after completion of the image processing for output and start of output of a dynamic image to an external apparatus.

Since parallel processing has a high processing load, it is preferable, for example, that the controller 101 control the output button 130e so as to be unable to be pressed (i.e., disable the output button 130e) during cine display in order that no bad influence may be exerted on output of a dynamic image for display to the external display apparatus 5. Further, it is preferable that the wireless IF 104 and the wireless IF 106 use different communication bands to prevent their communications from affecting one another.

In the image output control process shown in FIG. 3, cine display is started at the timing when the user instructs the display 103 to perform cine display, and in sync with (i.e., during) the cine display, a dynamic image for display is generated and output to the external display apparatus 5, so that the dynamic image for display can be promptly displayed on the large screen of the external display apparatus 5 at the timing desired by the user (e.g., when a plurality of medical staff members becomes available to check the dynamic image). The timing at which a dynamic image for display is output to the external display apparatus 5 is not particularly limited as far as it is after two or more frame images of a plurality of frame images of a dynamic image obtained by a series of steps of dynamic imaging are obtained. For example, a dynamic image for display may be generated in sync with cine display on the display 103, and started to be output to the external display apparatus 5 at the timing when cine display finishes. Alternatively, a dynamic image for display may be generated and output to the external display apparatus 5 at the timing when a series of steps of dynamic imaging finishes. The timing at which a dynamic image for display is output can be set in advance by the user operating the operation unit 102.

In the above, screen captures are generated as a dynamic image for display, but the dynamic image for display is not limited thereto. For example, the controller 101 may generate, as a dynamic image for display, a thinned (dynamic) image generated by thinning frame images of a dynamic image obtained by a series of steps of dynamic imaging or a low-resolution (dynamic) image composed of frame images into which frame images of a dynamic image obtained by a series of steps of dynamic imaging are converted, and as the second wireless output section 101b, output the thinned image or the low-resolution image to the external display apparatus 5 through the wireless IF 106.

Screen captures to be displayed on the external display apparatus 5 may be provided with additional information that is not displayed in the cine display screen 130. Examples thereof include the frame rate used for dynamic imaging, and the frame rate, the playback rate and the playback time used for fast-forward display.

The controller 101 as the second wireless output section 101b may determine whether a dynamic image for display needs to be output to the external display apparatus 5 on the basis of the content or settings of examination order information, and output the dynamic image for display to the external display apparatus 5 if the controller 101 determines that it is necessary. For example, if examination order information on dynamic imaging includes information indicating that it is for (during) an surgical operation, the controller 101 performs the output, whereas if the examination order information is for (during) a general round of visits (does not include the information indicating that it is for an surgical operation), the controller 101 does not perform the output. As another example, if examination order information on dynamic imaging is from a cardiovascular unit, the controller 101 performs the output, whereas if the examination order information is from an orthopedic unit, the controller 101 does not perform the output. The controller 101 may determine whether the output is necessary on the basis of the monitor size of the external display apparatus 5. For example, the controller 101 obtains information on the monitor size from the external display apparatus 5 at a predetermined timing, and if the monitor size is equal to or less than the monitor size of the display 103, does not perform the output because it is considered less urgent or necessary to perform display on the external display apparatus 5.

As another example, if body motion is detected or an image abnormality (that causes a problem in diagnosis or analysis, such as dose saturation or ROI loss) appears during preview display or cine display, the controller 101 stops generating screen captures and/or outputting the screen captures to the external display apparatus 5. Conversely, screen captures to be displayed on the external display apparatus 5 may be provided with information on detection of body motion or an image abnormality(ies) as matters to be noted.

As described above, the mobile radiographic imaging apparatus 10 includes the controller 101 that wirelessly outputs a first dynamic image including a plurality of frames obtained by dynamic imaging to an external apparatus, and after obtaining at least two frames of the plurality of frames of the first dynamic image, wirelessly outputs a second dynamic image for display based on the first dynamic image to the external display apparatus 5.

This enables output of a dynamic image for display to the external display apparatus 5 with the frame rate increased, and hence allows a doctor and/or others (i.e., user(s)) to promptly check a dynamic image on the external display apparatus 5 with the feeling of strangeness reduced.

For example, the controller 101 outputs the second dynamic image at a frame rate higher than a frame rate of the dynamic imaging. This allows a doctor and/or others to promptly check a dynamic image on the external display apparatus 5 with the feeling of strangeness reduced.

Further, for example, after generating frames for output of all the frames of the first dynamic image obtained by a series of steps of the dynamic imaging, the controller 101 starts to output the frames for output as the first dynamic image. Further, for example, before completing generating frames for output of all the frames of the first dynamic image obtained by a series of steps of the dynamic imaging, the controller 101 starts to output the second dynamic image.

This allows a doctor and/or others to check a dynamic image for display on the external display apparatus 5 before the controller 101 starts to output a dynamic image.

Further, for example, the controller 101 does not generate the second dynamic image during a series of steps of the dynamic imaging. This can reduce the processing load of the mobile radiographic imaging apparatus 10 and enables appropriate dynamic imaging.

More specifically, during the series of steps of the dynamic imaging, the controller 101 does not perform a process of generating, as the second dynamic image, screen captures that are obtained by capturing the screen of the display 103 in sync with the first dynamic image being played on the screen of the display 103. This can reduce the processing load of the mobile radiographic imaging apparatus 10 and enables appropriate dynamic imaging.

Further, after completing outputting the second dynamic image, the controller 101 outputs the first dynamic image. This can promptly output a dynamic image for display to the external display apparatus 5.

Further, the controller 101 outputs the second dynamic image based on at least one of (i) completion of the dynamic imaging, (ii) start of a display process of the first dynamic image on the display 103 and (iii) completion of the display process of the first dynamic image on the display 103. This allows a doctor and/or others to check a dynamic image for display at any of the above timings

Further, the controller 101 starts to generate the second dynamic image based on the first dynamic image during a series of steps of the dynamic imaging. This can move up output of a dynamic image for display to the external display apparatus 5.

Those described in the above embodiment and modification are some of preferred examples of the present disclosure, and hence the present disclosure is not limited thereto.

For example, in the above, the computer-readable storage medium storing the program(s) of the present disclosure is a hard disk, a nonvolatile semiconductor memory or the like, but not limited thereto and may be a portable recording medium, such as a CD-ROM. Further, as a medium to provide data of the program(s) of the present disclosure via a communication line, a carrier wave can be used.

The other detailed configurations and operations of the mobile radiographic imaging apparatus can be appropriately changed within a range not departing from the scope of the present disclosure.

Although one or more embodiments of the present disclosure have been described and illustrated in detail, the disclosed embodiments are made for purposes of illustration and example only and not limitation The scope of the present disclosure should be interpreted by terms of the appended claims

1. A mobile radiographic imaging apparatus capable of performing dynamic imaging of radiographic imaging, comprising a hardware processor that
wirelessly outputs a first dynamic image including a plurality of frames obtained by the dynamic imaging to an external apparatus, and
after obtaining at least two frames of the plurality of frames, wirelessly outputs a second dynamic image for display based on the first dynamic image to an external display apparatus.

2. The mobile radiographic imaging apparatus according to claim 1, wherein the hardware processor outputs the second dynamic image at a frame rate higher than a frame rate of the dynamic imaging.

3. The mobile radiographic imaging apparatus according to claim 1, wherein after generating frames for output of all the frames of the first dynamic image obtained by a series of steps of the dynamic imaging, the hardware processor starts to output the frames for output as the first dynamic image.

4. The mobile radiographic imaging apparatus according to claim 1, wherein before completing generating frames for output of all the frames of the first dynamic image obtained by a series of steps of the dynamic imaging, the hardware processor starts to output the second dynamic image.

5. The mobile radiographic imaging apparatus according to claim 1, wherein the hardware processor generates the second dynamic image based on the first dynamic image, but does not generate the second dynamic image during a series of steps of the dynamic imaging.

6. The mobile radiographic imaging apparatus according to claim 5, further comprising a display that displays the first dynamic image,
wherein the hardware processor
performs a predetermined type of image processing on the first dynamic image,
causes the display to play the image-processed first dynamic image on a screen of the display, and
during the series of steps of the dynamic imaging, does not perform a process of generating, as the second dynamic image, screen captures that are obtained by capturing the screen of the display in sync with the first dynamic image being played on the screen of the display.

7. The mobile radiographic imaging apparatus according to claim 1, wherein after completing outputting the second dynamic image, the hardware processor outputs the first dynamic image.

8. The mobile radiographic imaging apparatus according to claim 1, further comprising a display that displays the first dynamic image,
wherein the hardware processor outputs the second dynamic image based on at least one of (i) completion of the dynamic imaging, (ii) start of a display process of the first dynamic image on the display and (iii) completion of the display process of the first dynamic image on the display.

9. The mobile radiographic imaging apparatus according to claim 1, wherein the hardware processor starts to generate the second dynamic image based on the first dynamic image during a series of steps of the dynamic imaging.

10. A dynamic image output method that is performed by a mobile radiographic imaging apparatus capable of performing dynamic imaging of radiographic imaging, comprising:
wirelessly outputting a first dynamic image including a plurality of frames obtained by the dynamic imaging to an external apparatus; and
after obtaining at least two frames of the plurality of frames, wirelessly outputting a second dynamic image for display based on the first dynamic image to an external display apparatus.

11. A non-transitory computer-readable storage medium storing a program that causes, of a mobile radiographic imaging apparatus capable of dynamic imaging of radiographic imaging, a computer to:
wirelessly output a first dynamic image including a plurality of frames obtained by the dynamic imaging to an external apparatus; and
after obtaining at least two frames of the plurality of frames, wirelessly output a second dynamic image for display based on the first dynamic image to an external display apparatus.
